(19) United States
(12) Patent Application Publication
(10) Pub. No.: US 2018/0254104 A1
(43) Pub. Date: Sep. 6, 2018

(54) METHOD FOR PROVIDING EXERCISE CHECKUP SERVICE, TERMINAL DEVICE AND SERVER DEVICE FOR THE SAME

(71) Applicant: Suk Jea Hong, Seongnam-si, Gyeonggi-do (KR)

(72) Inventor: Suk Jea Hong, Seongnam-si, Gyeonggi-do (KR)

(21) Appl. No.: 15/755,375

(22) PCT Filed: Aug. 22, 2016
(86) PCT No.: PCT/KR2016/009247

§ 371 Date: Feb. 26, 2018

(30) Foreign Application Priority Data
Aug. 26, 2015 (KR) 10-2015-0120263
Aug. 5, 2016 (KR) 10-2016-0099781
Aug. 5, 2016 (KR) 10-2016-0099786

(51) Int. Cl. G16H 50/30; A61B 5/083; G16H 50/20; G16H 20/30

(52) U.S. Cl. CPC G16H 50/30; G16H 20/30; G16H 50/20; A61B 5/0833

(57) ABSTRACT

The present invention relates to a method for providing an exercise test service, a terminal device for the same, and a server device, and the method comprises the steps of: receiving an input of a test result value measured by performing, by an examinee, a test of predetermined conditions; calculating at least one diagnosis reference value, which indicates the cardiopulmonary capacity of the examinee, by using the inputted test result value; and sorting the examinee into one of a plurality of groups on the basis of at least one calculated diagnosis reference value, and providing information on an exercise program corresponding to the corresponding group.

### TECHNICAL FIELD

The present invention relates to a method for providing exercise checkup service for providing with an exercise program optimized to an examinee through cardiorespiratory functional capacity checkup.

### BACKGROUND ART

A variety of types of electronic devices are being developed and diffused due to development of electronic technology. In particular, a variety of types of display devices are being widely used such as television, cellphone, tablet PC, laptop PC, PC and the like. Accordingly, user's demand for being provided with a variety of services using a display device.

Particularly in these days, on a variety of exercises implemented in fitness, user become to be able to be provided with a variety of services including exercise instruction through web or mobile communication.

For an example, Korean laid-open patent gazette 2013-89765 proposes a personal exercise management system and a personal exercise management method. The proposed exercise management system comprises an exercise management server comprising an automatic member enlist portion enlisting automatically a user enlisting as a member at an alliance fitness center to a home page; an incoming and outgoing management portion receiving incoming and outgoing information of member's alliance fitness center; an instructor terminal notice portion noticing entered member's information to an instructor mobile terminal; an instructor exercise instruction result registration portion being registered with an exercise instruction result and body change result on the member from the instructor mobile terminal; and a member exercise result registration portion being registered with member's exercise result from a user terminal. And the member can be provided with instructor's exercise instruction service fit to self-exercise program based on a member's exercise information noticed to the instructor at real-time though visiting arbitrary alliance fitness center.

And recently, some attempts are continued for providing with exercise related services to user instead of trainers through mobile application or wearable device. However, they are in a level of providing with only fragmentary exercise effect such as exercise result and calorie consumption amount according to the same by detecting user's movement, by which there is a problem of decrease in service utilization.

### DISCLOSURE

### Technical Problem

An object of the present invention is to provide a method for providing exercise checkup service, a terminal device and a server device for the same by which an exercise program optimized to a user can be provided.

And another object of the present invention is to provide a wearable device and exercise strength display method thereof by which the strength of exercise that the user performs is visualized and efficiently displayed.

### Technical Solution

A method for providing exercise checkup service according to an embodiment of the present invention comprises being inputted with a checkup result value measured after an examinee performing a checkup of a predetermined condition; calculating at least one diagnosis reference value showing the examinee's cardiorespiratory functional capacity using the inputted checkup result value; and classifying the examinee into one of plurality of groups based on at least one of the calculated diagnosis reference value to provide with information on an exercise program corresponding to the classified group.

A terminal device according to an embodiment of the present invention comprises a user input portion being inputted with a checkup result value measured after an examinee performing a checkup of a predetermined condition; and a display portion displaying at least one diagnosis reference value showing the examinee's cardiorespiratory functional capacity calculated using the inputted checkup result value and displaying information on an exercise program corresponding to a group of plurality of groups in which the examinee being classified based on the diagnosis reference value.

And a server device according to an embodiment of the present invention comprises a communication portion providing an exercise checkup service in connection to a terminal device through a communication network and receiving a checkup result value measured after an examinee performing a checkup of a predetermined condition from the terminal device;

a diagnosis portion calculating at least one diagnosis reference value showing the examinee's cardiorespiratory functional capacity using the received checkup result value, and classifying the examinee into one of plurality of groups based on at least one of the calculated diagnosis reference value;

a control portion organizing an exercise program corresponding to the group in which the examinee being classified using information saved in the memory and making information on an exercise condition included in the organized exercise program be transmitted to the terminal device through the communication portion.

On the other hand, the method for providing exercise checkup service can be realized as a recording medium readable by a computer recording a program for being implemented on the computer.

### Advantageous Effects

According to an embodiment of the present invention, a checkup result value measured after running a predetermined distance or time on an indoor exercise device such as a treadmill, by which a diagnosis reference value showing the examinee's cardiorespiratory functional capacity is provided. Therefore, an objectified data on the examinee's cardiorespiratory functional capacity is provided with only input of the checkup result value using no high-priced checkup device.

And, an exercise checkup performed outdoor can be performed on indoor exercise device handily, by which not only the cost consumed for exercise checkup is saved but also the examinee's easiness can be improved.

And, the examinee is classified into one of plurality of groups based on the diagnosis reference value solved using the inputted checkup result value, and information on an exercise program corresponding to the classified group is provided, by which programs including exercise condition such as running speed and distance optimized to the user can be proposed.

According to another embodiment of the present invention, in a wearable device, current exercise strength can be displayed in a changeable color based on the heart rate reserve calculated using the user's age, resting heart rate and maximal heart rate and the user's current heart rate, by which the strength of exercise which the user performs is visualized to be shown efficiently.

### DESCRIPTION OF DRAWINGS

FIG. 1 is a flow chart showing a method for providing exercise checkup service according to an embodiment of the present invention.

FIG. 2 is a diagram for explanation of an embodiment for a method being inputted with a checkup result value measured after the examinee performs a checkup of a predetermined condition.

FIG. 3 is a diagram for explanation of an embodiment for indices of the examinee's cardiorespiratory functional capacity using the checkup result value.

FIG. 4 is a diagram for explanation of an embodiment on the examinee's level provided based on the index of cardiorespiratory functional capacity and exercise program information.

FIG. 5 is a diagram showing an example of an organization of checkup report of cardiorespiratory functional capacity provided as an output of exercise checkup according to an embodiment of the present invention.

FIG. 6 is a block diagram showing briefly an organization of system of exercise checkup service according to an embodiment of the present invention.

FIG. 7 is a block diagram showing an embodiment of an organization of terminal device shown in FIG. 6.

FIG. 8 is a block diagram showing an embodiment of an organization of server device shown in FIG. 6.

FIG. 9 to FIG. 11 are block diagrams showing other embodiments of organization of system of exercise checkup service according to the present invention.

FIG. 12 and FIG. 13 are diagrams showing examples of wearable device to which the present invention is applied.

FIG. 14 is a block diagram showing an organization of wearable device according to an embodiment of the present invention.

FIG. 15 is a flow chart showing exercise strength display method at a wearable device according to an embodiment of the present invention.

FIG. 16 is a block diagram showing an organization of wearable device according to another embodiment of the present invention.

FIG. 17 is a flow chart showing the exercise strength display method in a wearable device according to further another embodiment of the present invention.

### BEST MODE

Hereinafter, a method for providing exercise checkup service, a terminal device and a server device for the same are described in detail with reference to the attached drawings.

The present invention may be applied with a variety of change, and may have a variety of embodiments, in which a specific embodiment is illustrated in drawings and is described in detail. This is not to confine the present invention within a specific implement form, but all the changes, equivalents and substitutes can be understood to be included to the idea and technical range of the present invention.

The terminologies used in the present application are used only for description of specific embodiment, not of intention to confine the present invention. The expression of singularity can include the expression of plurality unless of which the meaning is explicitly differently set in context.

In the present invention, the term of ‘include’ or ‘have’ or the like is to designate the existence of feature, number, step, operation, constitution factor, part or combination thereof written in the specification, and the existence or possibility of addition of one or more other features, numbers, steps, operations, constitution factors, parts or combinations thereof is understood to be not excluded in advance.

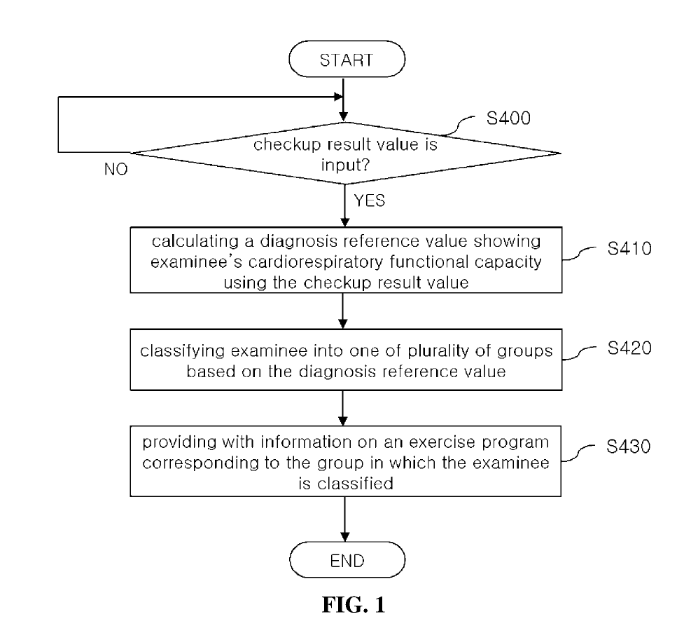

FIG. 1 shows a flow chart of a method for providing exercise checkup service according to an embodiment of the present invention. Hereinafter, the present invention will be described with an example that the device providing an exercise checkup service performs the method for providing exercise checkup service according to an embodiment of the present invention, while the present invention is not limited to such, and the method for providing exercise checkup service to be described in reference to FIG. 1 hereinafter may be performed by a device or two or more devices performing function except for the exercise checkup as a main function.

Referring to FIG. 1, the device providing exercise checkup service is inputted with a checkup result value measured after the examinee performs a checkup of a predetermined condition (S400).

The checkup may be any one of plurality of checkups predetermined to be performed in different conditions one another, which may be one for measuring time or distance after the examinee runs for predetermined distance or time outdoor or desirably on an indoor exercise device such as treadmill.

For example, a checkup person processing the checkup or the examinee can select any one of ‘2.4 km Run Test’, ‘12 min Run Test’, ‘M-Bruce Protocol’ and ‘Balke Protocol’ to perform the corresponding checkup.

The ‘2.4 km Run Test’ is an exercise checkup for running distance of 2.4 km at a shortest time, and in case of indoor checkup, the examinee (or checkup person) may start at a proper speed on a treadmill and control the speed to a pace fit to the examinee's maximum capacity value and can measure running time after finishing 2.4 km.

The ‘12 min Run Test’ is an exercise checkup for running as much distance as possible for 12 minutes, and in case of indoor checkup, the examinee (or checkup person) may start at a proper speed on a treadmill, may control speed, and can measure the running distance according to the examinee's maximum capacity value.

On the other hand, ‘M-Bruce Protocol’ is for running at a speed of about 5.3 km/h on a gradient of 11 degree, and after start, stage may be increased every 1 minutes as shown in table 1 below, and time until the examinee stops can be measured.

TABLE 1

| | Stage | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Speed (km/h) | 3.7 | 4.0 | 4.6 | 5.0 | 5.4 | 5.9 | 6.4 | 6.7 | 7.2 | 7.7 | 8.0 | 8.3 |
| Gradient | 11 | 12 | 12 | 13 | 14 | 14 | 15 | 16 | 16 | 17 | 18 | 18 |

And, in ‘Balke Protocol’, the running speed is fixed at about 5.3 km/h, and the gradient is increased 1 degree in every 1 minutes from 0 degree, and time until the examinee stops can be measured.

The checkup method as described above may be selected according to the examinee's exercise capacity, and for example, the lower the examinee's exercise capacity, ‘2.4 km Run Test’, ‘12 min Run Test’, ‘M-Bruce Protocol’, and ‘Balke Protocol’ may be applied in order.

The checkup methods described above is an example of checkup method for providing exercise checkup service according to an embodiment of the present invention, to which the present invention is not limited, while a variety of exercise checkup method may be applied which can checkup the examinee's cardiorespiratory functional capacity.

And, to checkup the examinee's maximum capacity value, any one of checkup methods described above is performed several times repeatedly, or two or more methods are mixed, by which through a checkup method suitable for the examinee, the checkup result value can be measured.

For example, utilizing a checkup result value measured through a checkup of one time, and utilizing a verified increase formula (for example, increasing the running speed by 0.3 km/h), with repeating checkup several times, maximum value for the examinee can be sought.

Accordingly, in case that the checkup person is an expert, an optimum value for the checkup result value of a examinee can be sought through one or two times of checkup, while in case that the checkup person is a non-expert, more times of checkup can be repeated to seek the checkup result value for the examinee. And, the checkup as described above may facilitate an exercise effect by only the repeated checkup itself.

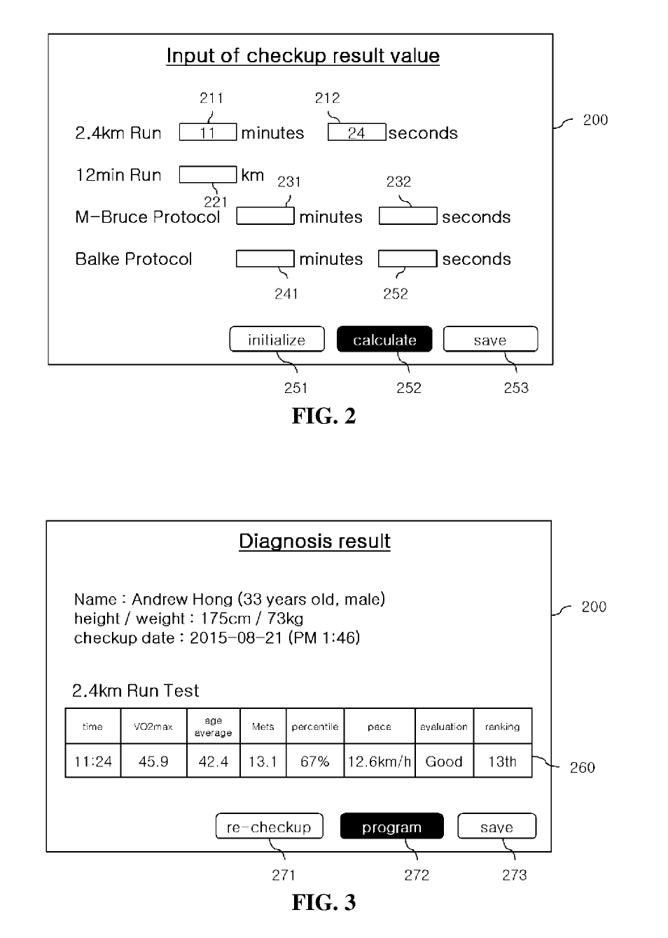

The checkup result value measured by the above described method may be inputted through a screen 200 as shown in FIG. 2. Referring to FIG. 2, there is displayed checkup methods which the examinee can perform, and regions where the checkup result value measured by each of the checkup method is inputted may be shown on the screen 200 of the device providing the exercise checkup service.

For example, in case that the examinee performs ‘2.4 km Run Test’ and finishes 2.4 km in ‘11 minutes 24 seconds’ as a result, the above time may be inputted in the ‘2.4 km Run Test’ result value input boxes 211 and 212 on the screen 200.

And, on the screen shown in FIG. 2, ‘initialize’ button 251 may be selected to initialize the checkup result value inputted on the screen 200, ‘calculate’ button 252 may be selected to request calculation of diagnosis reference value according to the inputted checkup result value, or ‘save’ button 253 may be pressed to save the inputted checkup result value on the screen 200 into a memory of inside of device providing exercise checkup service or external database (DB).

When the input of the checkup result value is verified (S400), the device providing the exercise checkup service may calculate at least one diagnosis reference value showing the examinee's cardiorespiratory functional capacity using the inputted checkup result value (S410).

For example, the device providing the exercise checkup service can calculate the examinee's maximum oxygen uptake (VO2max) with the diagnosis reference value using the inputted checkup result value, and according to the calculated maximum oxygen uptake (VO2max), the examinee's oxygen utilization rate (Mets) can be solved.

To show the index of the examinee's cardiorespiratory functional capacity, the diagnosis result value 260 calculated as above may be displayed on the screen 200 of the device providing exercise checkup service with information on the examinee as shown in FIG. 3. Referring to FIG. 3, the examinee information may include name, age, sex, height, weight, and the like, which are inputted information in advance before calculation of diagnosis reference value.

And, the result comparing the calculated diagnosis reference value and the examinee's age average value is displayed as a ranking or percentile, and the examinee's cardiorespiratory functional capacity evaluated according to the comparison result may be displayed as one of ‘Superior, ‘Excellent’, ‘Good’, ‘Fair’, ‘Poor’ and ‘Very Poor’.

For example, in case that the percentile value calculated by comparing the examinee's maximum oxygen uptake (VO2max) to the age average value is 95% or over, it may be determined as ‘Superior, between 80% and 95%, ‘Excellent’, between 60% and 80%, ‘Good’, between 40% and 60%, ‘Fair’, between 20% and 40%, ‘Poor’, and between 0% and 20%, ‘Very Poor’.

And, on the screen shown in FIG. 3, selecting the ‘re-checkup’ button 261 may move the screen to the screen shown in FIG. 2 where the checkup result value can be inputted after re-checkup, selecting the ‘program’ button 262 may demand to the examinee an optimized exercise program information based on the calculated diagnosis reference value, or pushing the ‘save’ button 263 may save the diagnosis reference values displayed on the screen 200 into a internal memory or external database (DB).

After that, the device providing exercise checkup service may classify the examinee into one of plurality of groups (S420) based on the diagnosis reference value calculated at S410, and may provide with information on the exercise program corresponding to the group in which the examinee is classified (S430).

That is, there may exist a plurality of groups in which the examinee may be classified according to the cardiorespiratory functional capacity level, and the plurality of groups are preset as to correspond to each of plurality of exercise programs having different exercise condition one another.

For example, the examinee may be classified to any one of ‘beginner’, ‘intermediate’ and ‘advanced’ according to that the oxygen utilization rate (Mets) or maximum oxygen uptake (VO2max) value belongs to what range, and the exercise programs each corresponding to ‘beginner’, ‘intermediate’ and ‘advanced’ may be different one another in exercise condition, that is running speed and distance proposed to the examinee.

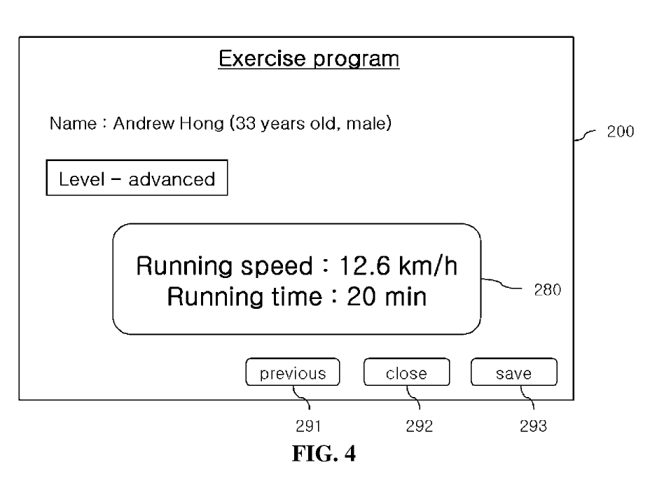

In case that the examinee's oxygen utilization rate (Mets) calculated according to checkup result value is 12 or more, as shown in FIG. 4, examinee's cardiorespiratory functional capacity level may be classified as ‘advanced’ and may be displayed on the screen 200.

And, on the screen 200, information 280 on the exercise program including the exercise condition (running speed and running time) corresponding to ‘advanced’ may be displayed to be provided to the examinee.

According to an embodiment of the present invention, the running speed included in the exercise program information 280 may be determined in consideration of the examinee's average running speed, which may be a Pace value calculated using the checkup result value.

On the other hand, the running time included in the exercise program information 280 may be determined in consideration of calorie amount to be consumed through the exercise.

Pressing the ‘save’ button 293 on the screen shown in FIG. 4 may save the examinee's level displayed on the screen 200 and information on the exercise program proposed corresponding thereto into an internal memory of the device providing exercise checkup service or external database (DB).

According to another embodiment of the present invention, only by inputting the examinee's checkup result value, the inputted checkup result value, at least one diagnosis reference value calculated as above, and information on the exercise program corresponding to the group where the examinee is classified are displayed on the screen 200 or outputted on a paper or the like together to be provided to the examinee (or checkup person).

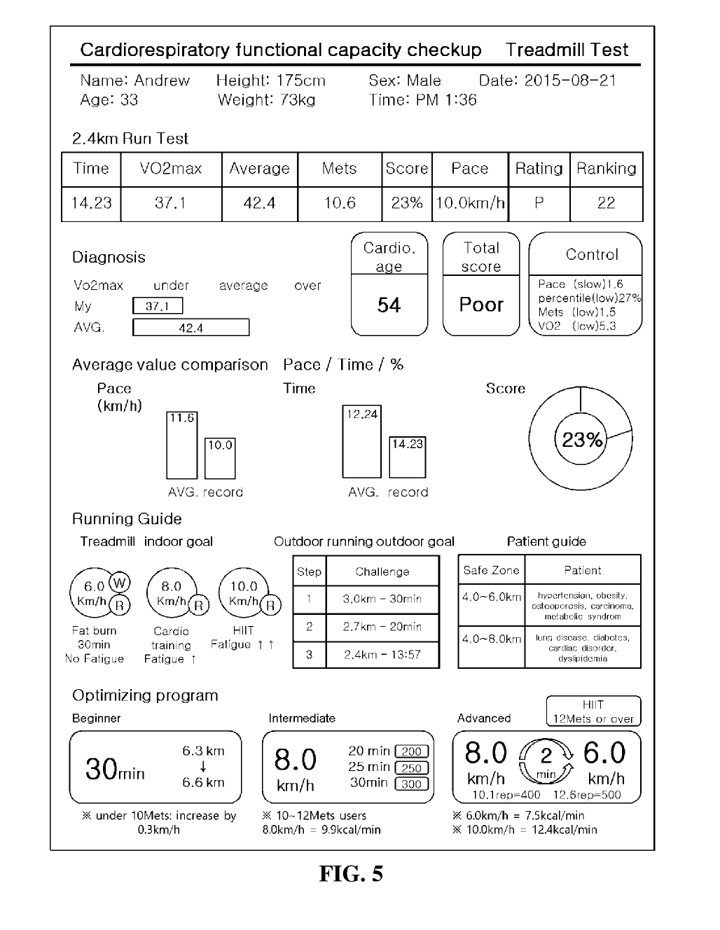

FIG. 5 shows an examination on an organization of cardiorespiratory functional capacity checkup report provided as a result of exercise checkup according to an embodiment of the present invention, wherein description on the same ones as those described in reference to FIG. 1 to FIG. 4 in the organizations of report shown in FIG. 5 will be omitted hereinafter.

Referring to FIG. 5, on top of the cardiorespiratory functional capacity checkup report provided to the examinee (or checkup person), checkup date and time may be displayed together with examinee's name, age, height, weight, and sex.

Below of the examinee information, the checkup (for example, ‘2.4 km Run Test’) result value (Time) and the maximum oxygen uptake (VO2max) calculated using the corresponding checkup result value may be displayed together with an average value (Average) of the same sex and age as the examinee.

And, as an index of diagnosis showing the examinee's cardiorespiratory functional capacity, the oxygen utilization rate (Mets) calculated in proportion to the maximum oxygen uptake (VO2max), the percentile of the maximum oxygen uptake (Score) in reference to the examinee's sex/age, the pace (Pace) showing the examinee's average running speed when checkup, the evaluation of the maximum oxygen uptake (VO2max) (Rating) in reference to the examinee's sex/age, and the maximum oxygen uptake ranking (Ranking) among the same sex and age as the examinee from examinees performed the exercise checkup may be displayed.

On the other hand, on a center part of the cardiorespiratory functional capacity checkup report, the examinee's cardiorespiratory functional capacity indices displayed on the upper part may be displayed visually, and at ‘diagnosis’ item, examinee's maximum oxygen uptake (VO2max), pace (Pace), percentile (Score), and result and difference by comparison of oxygen utilization rate (Mets) and sex/age average value may be shown visually. For example, according to the comparison result, some example for control method may be displayed together with total score on the examinee's cardiorespiratory age, and cardiorespiratory capacity.

And, at ‘average value compare’ item, the result and percentile (Score) comparing examinee's pace (Pace) and checkup result value of time (Time) to sex/age average value may be displayed visually.

At lower part of the cardiorespiratory functional capacity checkup report, according to the examinee's cardiorespiratory functional capacity level, information on the exercise program proposed to the examinee may be displayed.

For example, at ‘running method’ item, the running method proper to the examinee may be displayed based on the examinee's cardiorespiratory functional capacity index calculated at upper part, wherein the running method may be organized with a plurality of exercise conditions according to different exercise strength or exercise step each other.

More specifically, as an exercise of highest strength (HIIT), ‘10.0 km/h’ of the same running speed as that of pace (Pace) calculated at upper part may be displayed, as an exercise of middle strength (cardio training), ‘8.0 km/h’ of running speed corresponding to about 80% of pace (Pace) may be displayed, and as an exercise of low strength (Fat burn 30 min), ‘6.0 km/h’ of running speed corresponding to about 60% of pace (Pace) may be displayed.

And, information on the running distance and time challengeable by step, and information on safe exercise condition (running speed) according to illness type may be displayed at ‘running method’ item.

At ‘optimization program’ item, the group where the examinee is classified may be displayed based on the examinee's cardiorespiratory functional capacity index calculated at upper part, and may be provided with information on the exercise program proper to the examinee's group.

More specifically, the examinee's group may be determined according to the oxygen utilization rate (Mets) calculated at upper part, and for example, in case that the oxygen utilization rate (Mets) is ‘10’ under, it may be classified as ‘beginner’, in case that it is between ‘10’ and ‘12’, as ‘intermediate’, and in case that it is ‘12’ or over, as ‘advanced’.

On the other hand, at ‘optimization program’ item, information on the exercise program each corresponding to ‘beginner’, ‘intermediate’ and ‘advanced’ may be displayed.

In case of ‘beginner’, an exercise program for running at a running speed corresponding to 60% of pace (Pace) calculated at upper part for 30 minutes may be displayed, and at every time of success, increase of running speed by 0.3 km/h may be proposed.

In case of ‘intermediate’, an exercise program for running at a running speed corresponding to 80% of pace (Pace) may be displayed, and different running time may be proposed one another according to calorie amount to be consumed.

On the other hand, in case of ‘advanced’, an exercise program of repeating running 2 minutes at a running speed corresponding to 100% of pace (Pace), and after that running another 2 minutes at a running speed corresponding to 60% of pace (Pace) may be displayed, and different repetition number may be proposed one another according to calorie amount to be consumed.

As described above, according to the method for providing exercise checkup service according to an embodiment of the present invention, a checkup result value measured by running for predetermined distance or time on an indoor exercise device like treadmill is inputted, and a diagnosis reference value showing the examinee's cardiorespiratory functional capacity is provided, by which without using a high-priced checkup device and by input of only checkup result value, an objectified date on the examinee's cardiorespiratory functional capacity can be provided.

And, the examinee is classified into one of plurality of groups based on the diagnosis reference value solved using the inputted checkup result value, and information on the exercise program corresponding to the classified group is provided, by which a program including the exercise condition such as running speed and distance and the like optimized to the user can be proposed.

On the other hand, the method for providing exercise checkup service according to an embodiment of the present invention may be realized as an exercise checkup system which will be described below. The description may be omitted for the same organization and each of operations of the exercise checkup system to be described below as that of described in reference to FIG. 1 to FIG. 5.

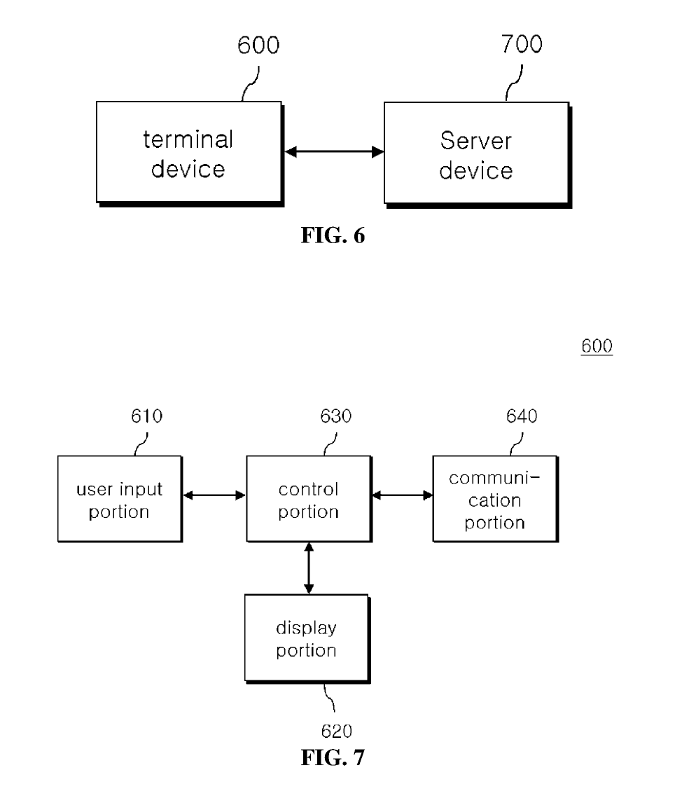

FIG. 6 shows the organization of the exercise checkup service system according to an embodiment of the present invention as a block diagram, and the depicted exercise checkup service system may be organized including a terminal device 600 and a server device 700.

Referring to FIG. 6, the terminal device 600 can perform method for providing exercise checkup service as described in reference to FIG. 1 to FIG. 5 alone or in connection with the server device 700.

For example, the terminal device 600 may be inputted with a checkup result value measured after the examinee performs the checkup of a predetermined condition, may calculate at least one diagnosis reference value showing the examinee's cardiorespiratory functional capacity using the inputted checkup result value, may classify the examinee into one of plurality of groups based on the calculated diagnosis reference value, and may provide with information on the exercise program corresponding to the classified group.

On the other hand, the terminal device 600 may perform only the role to be inputted with a checkup result value measured after the examinee performs a checkup of predetermined condition, and to display information on the exercise program corresponding to the diagnosis reference value calculated according to the same and the examinee group, while the role to calculate at least one diagnosis reference value showing the examinee's cardiorespiratory functional capacity using the inputted checkup result value, and to classify the examinee into one of plurality of groups may be performed by the server device 700.

The terminal device according to the present invention may include desktop computer, cellphone, smart phone, laptop computer, terminal device for digital broadcasting, PDA (Personal Digital Assistants), PMP (Portable Multimedia Player), Navigation device and the like, but not limited to such, but may be a variety of other devices capable of user input and information display and the like.

Referring to FIG. 7, the terminal device 600 may be organized to comprise a user input portion 610, a display portion 620, a control portion 630 and a communication portion 640.

The user input portion 610 of the terminal device 600 may be inputted with the checkup result value measured after the examinee performs the checkup of predetermined condition.

On the other hand, the display portion 620 may display at least one diagnosis reference value showing the examinee's cardiorespiratory functional capacity calculated using the inputted checkup result value, and may display information on the exercise program corresponding to the group where the examinee is classified based on the diagnosis reference value from the plurality of groups.

The control portion 630 may have the checkup result value inputted through the user input portion be transmitted 610 through the communication portion 640 to the server device 700, and may have information on the exercise program corresponding to the group where the examinee is classified which is received from the server device 700 through the communication portion 640 be displayed through the display portion 620, and may perform other role to control the whole operation of the terminal device 600.

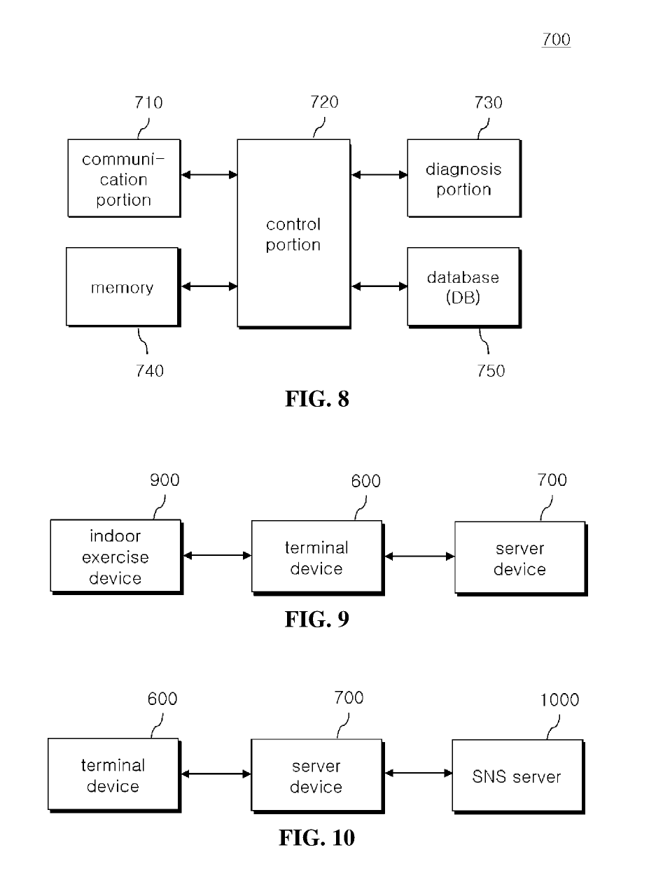

Referring to FIG. 8, the server device 700 may be organized to comprise a communication portion 710, a control portion 720, a diagnosis portion 730, a memory 740 and a database (DB) 750.

The server device 700 may be provided with the communication portion 710 to provide with an exercise checkup service in connection to the terminal device 600 through the communication network, and the communication portion 710 may receive the checkup result value measured after the examinee performs the checkup of predetermined condition from the terminal device 600.

The control portion 720 may transfer the checkup result value received through the communication portion 720 to the diagnosis portion 730, and the diagnosis portion 730 may calculate at least one diagnosis reference value showing the examinee's cardiorespiratory functional capacity using the inputted checkup result value.

And, the diagnosis portion 730 may classify the examinee into one of plurality of groups based on the calculated diagnosis reference value.

On the other hand, in the memory 740, information on the plurality of exercise programs each corresponding to a plurality of groups where the examinee is classified, and the plurality of exercise programs may have each of different exercise condition (for example, running speed and time) one another.

The control portion 720 may organize the exercise program corresponding to the group where the examinee is classified using information saved in the memory 740, and may have information on the exercise condition included in the organized exercise program be transmitted to the terminal device 600 through communication portion 710.

The organization of exercise checkup service system described in reference to FIG. 6 to FIG. 8 is only an embodiment of the present invention, to which the present invention is not limited to such, but the exercise checkup service system according to the present invention may have an organization of another embodiment of the present invention as shown in FIG. 9 to FIG. 11.

Referring to FIG. 9, the terminal device 600 may be connected to the indoor exercise device 900 such as treadmill using a wired or wireless communication method, and may be inputted with the checkup result value measured after the examinee performs the checkup of predetermined condition by receiving directly from the indoor exercise device 900.

And, the terminal device 600 may transmit information on the exercise program corresponding to the group where the examinee is classified to the indoor exercise device 900, and may have the exercise program (for example, running speed and time) optimized to the examinee be set automatically to the indoor exercise device 900 without any further input manipulation.

Referring to FIG. 10, the server device 700 may be connected to a SNS (Social Network Service) server 1000 using the communication network to share examinee's checkup result value, diagnosis reference values calculated using the checkup result value, or information on the exercise program optimized to the examinee through SNS.

And, as shown in FIG. 11, the server device 700 may be connected to a near field wireless communication device 1100 installed at a specific position using the communication network, and may have the checkup result value be calculated automatically at the server device 700 based on the examinee's position and time information acquired according to information received from the near field wireless communication device 1100.

In this case, the near field wireless communication device 1100 may be installed in plurality at a specific position of outdoor, and each of the near field wireless communication device 1100 may acknowledge the position of the examinee through the near field wireless communication with the mobile terminal which the examinee holds to transmit the corresponding position information to the server device 700.

For example, the near field wireless communication between the near field wireless communication device 1100 and the examinee's mobile terminal as described above may be a Bluetooth Low Energy (BLE) communication. The Bluetooth Low Energy is a wireless communication protocol used in transmit a message at low energy, and the BLE specification is defined in Volume 6 of the Bluetooth Specification.

Such a Bluetooth Low Energy may be operated at 2.4 GHz ISM band (2400-2483.5 MHz) which is a wireless transmit frequency of short wavelength, and may use 40 RF channel of 2 MHz within the range. And, the Bluetooth Low Energy may use a wireless technology called as frequency-hopping spread spectrum method in which the data to be transmitted is cut finely, and the cut lump may be transmitted through different channels.

And, the Bluetooth Low Energy transmission may have transmission range of about 50 m, data rate of 1 Mb/s, and consumption power of 1%˜50% comparing to conventional Bluetooth.

However, the near field wireless communication between the near field wireless communication device 1100 described below and the mobile terminal may be realized using a variety of near field wireless communication specification such as ZigBee, or Wi-Fi or the like.

On the other hand, according to an embodiment of the present invention as described in reference to FIG. 1 to FIG. 11, an exercise method optimized to the user's exercise capacity, more specifically to the cardiorespiratory functional capacity may be proposed, and the exercise method optimized to the user like such may be improved in its effect by inducing the user to continue to exercise with the exercise strength corresponding thereto.

For the above, according to another embodiment of the present invention, to show efficiently the strength of exercise the user performs in visualization, user's current exercise strength may be displayed with color changed corresponding thereto at a wearable device which the user wore at the time of exercise.

For example, the current exercise strength may be displayed with a changeable color at the wearable device based on the calculated heart rate reserve and user's current heart rate using the user's age, resting heart rate and maximal heart rate, by which the user can verify the color displayed on the wearable device, and can continue to perform exercise at an exercise strength proper to cardiorespiratory functional capacity of oneself.

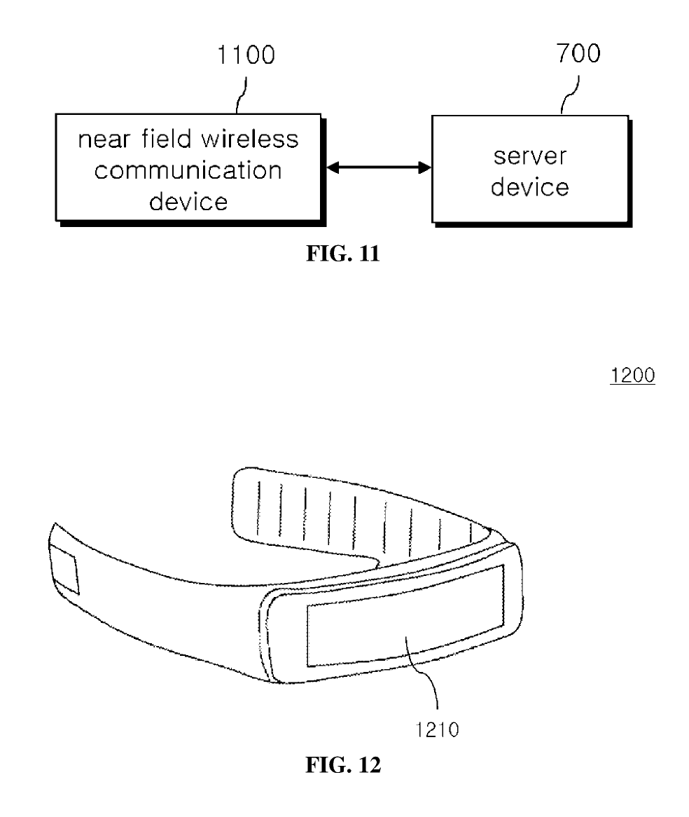

The wearable device may mean a variety of devices which can be wore on the user's body, and as shown in FIG. 12, may be provided with a display portion 1210 capable of displaying text, image and a variety of colors, and may have a shape to be fixed temporarily on the user's wrist.

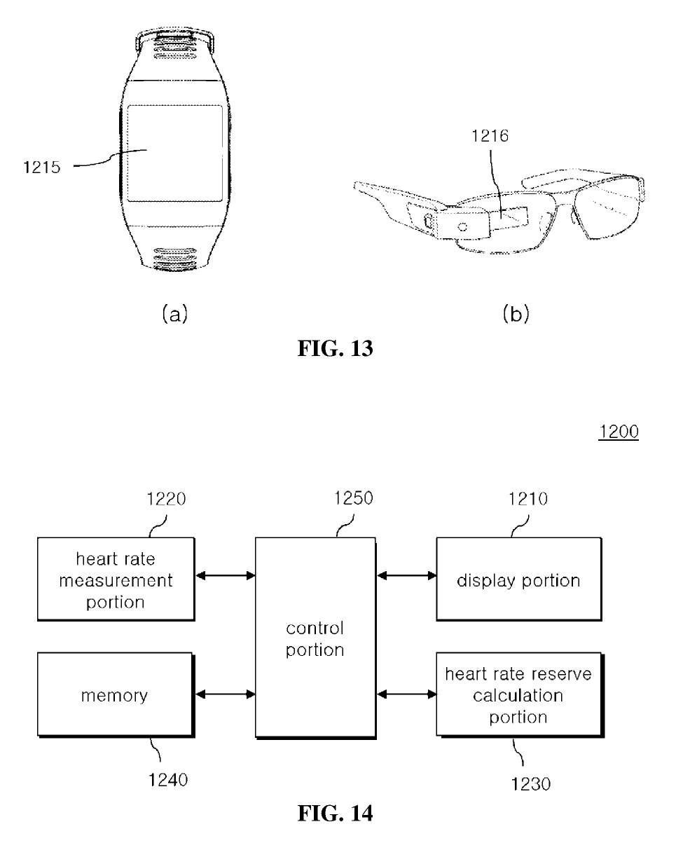

A wearable device 1200 according to the present invention may be realized in not only a form of smart band, but also a variety of form, and for example, may have a form of smart watch provided with a display portion 1215 on the front face as shown in (A) of FIG. 13, or may have a form of smart glass provided with a display portion 1216 at front face of the lens as shown (B) of FIG. 13.

FIG. 14 is showing an organization of the wearable device according to an embodiment of the present invention as a block diagram, and depicted wearable device 1200 may be organized by comprising a display portion 1210, a heart rate measurement portion 1220, a heart rate reserve calculation portion 1230, a memory 1240 and a control portion 1250.

Referring to FIG. 14, the display portion 1210 provided at the wearable device 1200 may be for displaying the user's exercise strength, and may display the user's current exercise strength in a color corresponding thereto to visualize and transfer to the user.

The heart rate measurement portion 1220 may measure the user's heart rate to transfer to the control portion 1250, while the heart rate reserve calculation portion 1230 may calculate the user's heart rate reserve using the user's age, resting heart rate and maximal heart rate.

On the other hand, information on the user's age, resting heart rate and maximal heart rate may be saved in the memory 1240 in advance, and information on the user's heart rate reserve calculated at the heart rate reserve calculation portion 1230 may also be saved in the memory 1240.

For example, the user's heart rate may be measured continuously through the heart rate measurement portion 1220 while the user wears the wearable device 1200 and does an everyday life or does exercise or the like, and information on the heart rate measured continuously as above may be saved in the memory 1240.

In this case, corresponding user's resting heart rate and maximal heart rate may be detected based on the user's past heart rates measured in predetermined period or predetermined number and saved in the memory 1240, and information on the user's resting heart rate and maximal heart rate detected as above may be saved in the memory 1240 in advance.

The control portion 1250 may change color displayed on the display portion 1210 to show the corresponding user's exercise strength based on the user's current heart rate measured continuously through the heart rate measurement portion 1220 and the user's heart rate reserve calculated through the heart rate reserve calculation portion 1230.

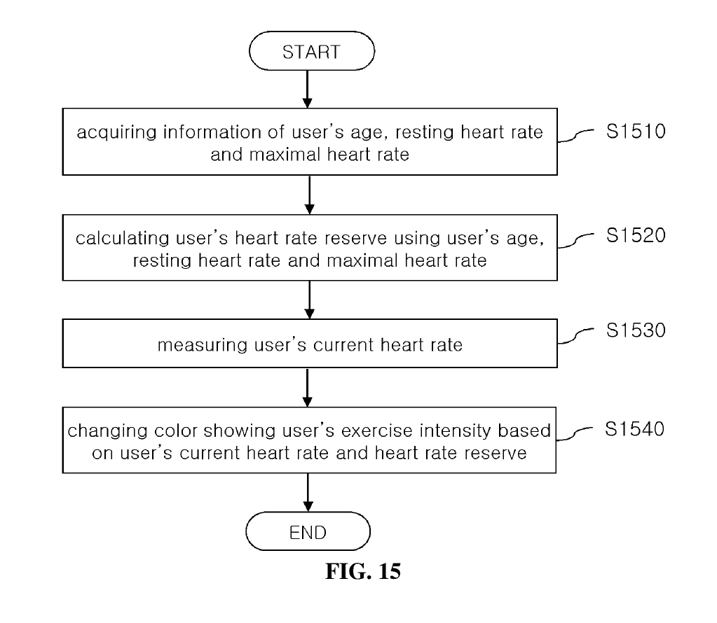

FIG. 15 is showing an exercise strength display method on a wearable device according to an embodiment of the present invention as a flow chart, and the depicted exercise strength display method will be described in connection with the block diagram showing the organization of the wearable device according to an embodiment of the present invention shown in FIG. 14.

Referring to FIG. 15, first, the heart rate reserve calculation portion 1230 of the wearable device 1200 may acquire information on the user's age, resting heart rate and maximal heart rate (S1510), and may calculate the user's heart rate reserve using the acquired user's age, resting heart rate and maximal heart rate (S1610).

For example, the heart rate reserve calculation portion 1230 may read in information on the corresponding user's resting heart rate and maximal heart rate from the memory 1240 through the control portion 1250, and as in equation 1 below, can calculate the heart rate reserve (H_margin) by subtracting user's age (Age) and resting heart rate (H_stable) from maximal heart rate (H_max).

H__{^{margin}}=H__{^{max}}−AGE−H__{^{stable}} (1)

The heart rate measurement portion 1220 may measure the user's current heart rate (h) who wears the wearable device 1200 and is exercising (S1530).

For example, the heart rate measurement portion 1220 may measure the user's heart rate continuously at a predetermined interval using a heart rate measurement sensor provided on the wearable device 1200 to be contact to the user's body (for example, skin).

After that, the control portion 1250 may change color displayed on the display portion 1210 to show the corresponding user's exercise strength based on the user's current heart rate measured continuously through the heart rate measurement portion 1220 and the user's heart rate reserve calculated at the heart rate reserve calculation portion 1230 (S1540).

For example, the control portion 1250 may calculate a target heart rate using the user's heart rate reserve (H_margin) and a preset exercise strength, and may determine color displayed on the display portion 1210 according to the result of comparison of the calculated target heart rate and the user's current heart rate.

Here, the exercise strength may be a target exercise strength set by selection in advance by the user before exercise, and more specifically, as shown in table 2 below, the exercise strength may be selected by selection of any one exercise program from the user through a user input portion (not shown) from a plurality of exercise programs divided in plurality of sections.

TABLE 2

| | | | | cardiopulmonary |
|---|---|---|---|---|
| Exercise | | | | function |
| program | Walking | Safe | Diet | (Training) |
| Exercise | 20~30 | 30~40 | 40~60 | 60~80 |
| strength (%) | | | | |

Referring to table 2, there may exist a plurality of exercise programs having different exercise strength one another, for example, ‘Walking exercise mode’, ‘Safe exercise mode’, ‘Diet exercise mode’ and ‘cardiorespiratory function (Training) exercise mode’, and the user may select an exercise program according to the desired exercise strength from the plurality of exercise programs before start of exercise.

On the other hand, when the target exercise strength (ES) is set according to the exercise program selected by the user, the user's target heart rate (H_target) may be calculated using the user's heart rate reserve (H_margin), target exercise strength (ES) and resting heart rate (H_stable) as in equation 3 below.

H__{^{target}}=H__{^{marin}}*ES+H__{^{stable}} (2)

When the user's target heart rate (H_target) is calculated using equation 2 according to the exercise strength by the exercise program shown in table 2, the range of target heart rate (H_target) on each of plurality of exercise programs may be solved as in table 3.

TABLE 3

| | | | | cardiopulmonary |
|---|---|---|---|---|
| Exercise | | | | function |
| program | Walking | Safe | Diet | (Training) |
| Exercise | 20~30 | 30~40 | 40~60 | 60~80 |
| strength (%) | | | | |
| target heart rate | 96~109 | 109~122 | 122~148 | 148~174 |
| (per minute) | | | | |

In case shown in table 3, in case that the user selects ‘Walking mode’ from plurality of exercise programs, the heart rate between 96˜109 per minute may be set as the target exercise strength range, in case of ‘Safe mode’, 109˜122, in case of ‘Diet mode’, 122˜148, and in case of ‘cardiorespiratory function mode’, 148˜147.

On the other hand, the plurality of exercise programs selectable by the user and the exercise strength corresponding thereto are described as preset in the description above, to which the present invention may not be limited, but the user may set directly a ‘Custom mode’ of exercise program for oneself having a specific exercise strength.

In this case, the control portion 1250 may determine color displayed on the display portion 1210 according to whether the user's current heart rate is in the target exercise strength range corresponding to a specific exercise program (for example, exercise program which the user selected).

After that, the control portion 1250 may verify whether the user's current heart rate (h) measured through the heart rate measurement portion 1220 is in the range (h1˜h2) of the target heart rate calculated at above S1610 (S1620).

In case that, as a result of the verification, the user's current heart rate (h) is in the range (h1˜h2) of the target heart rate, the control portion 1250 may have a first color be displayed on the display portion 1210 (S1630).

For example, in case that the user's current heart rate (h) is in the range (h1˜h2) of the target heart rate, the control portion 1250 may control the display portion 1210 to display (or blink) ‘Blue’ which may be a color showing the exercise strength proper.

On the other hand, as a result of the verification, the user's current heart rate (h) is not in the range (h1˜h2) of the target heart rate, the control portion 1250 may verify again whether the user's current heart rate (h) is smaller than that of lowest value (h1) of target heart rate range (S1640).

In case that the user's current heart rate (h) has smaller value than the lowest value (h1) of target heart rate range, the control portion 1250 may have a second color be displayed on the display portion 1210 (S1650).

For example, in case that the user's current heart rate (h) is smaller than the lowest value (h1) of the target heart rate, the control portion 1250 may control the display portion 1210 to display (or blink) ‘Green’ which is a color showing the exercise strength weak.

On the contrary, the user's current heart rate (h) have a larger value than the maximum value (h2) of the target heart rate range, the control portion 1250 may have a third color be displayed on the display portion 1210 (S1650).

For example, in case that the user's current heart rate (h) is larger than the maximum value (h2) of the target heart rate, the control portion 1250 may control the display portion 1210 to display (or blink) ‘Red’ which may be color showing the exercise strength excessive.

In case that the user selects ‘Diet exercise mode’ from plurality of exercise programs shown in table 2 and table 3 to perform running exercise, the range of the target heart rate may be set to 122˜148 times per minute according to the exercise strength range to which the user is targeting.

The wearable device 1200 according to an embodiment of the present invention may further be provided with a vibration generation portion (not shown) or a voice output portion (not shown), by which in case that the user's current heart rate is out of the target exercise strength range (or, out of the target heart rate range), a notice may be provided to the user by vibration or voice.

Though an embodiment of the present invention was described with an example that showing ‘proper’, ‘weak’, and ‘excessive’ of the user's current exercise strength is by use of ‘Blue’, ‘Green’, and ‘Red’, to which the present invention is not be limited, but the colors according to the current exercise strength can be changed or set by user arbitrarily.

According to another embodiment of the present invention, the strength in which the user is currently exercising may be visualized in color and displayed based on the target exercise speed calculated based on the user's cardiorespiratory functional capacity at the wearable device and the user's current speed.

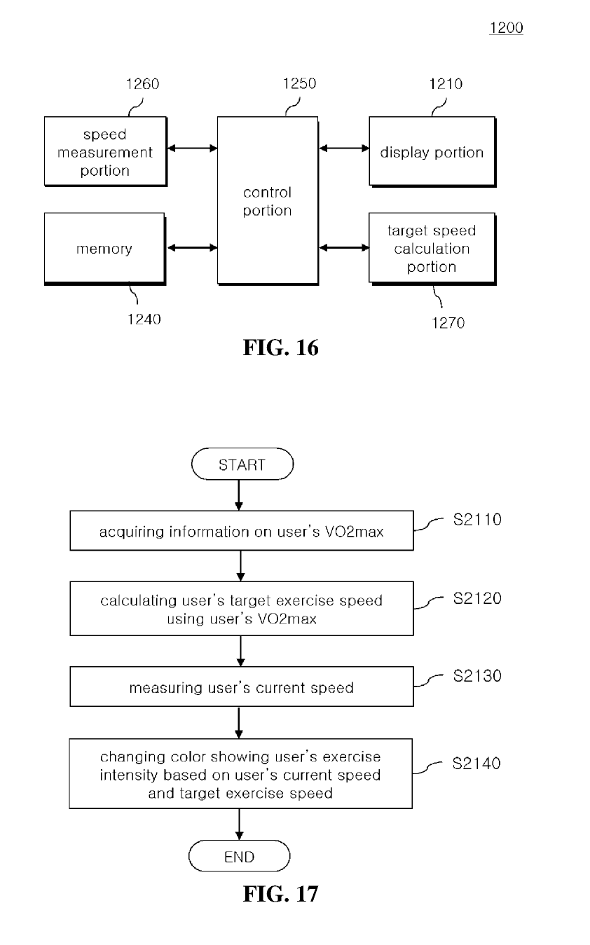

FIG. 16 is showing an organization of the wearable device according to another embodiment of the present invention as a block diagram, and the depicted wearable device 1200 may be organized by including a display portion 1210, a speed measurement portion 1260, a target speed calculation portion 1270, a memory 1240 and a control portion 1250.

The description on organizations of the wearable device 1200 shown in FIG. 16 and operation thereof may be omitted hereinafter for the same ones described in reference to FIG. 14 and FIG. 15.

Referring to FIG. 16, the speed measurement portion 1260 may measure the user's speed to transfer to the control portion 1250, and the target speed calculation portion 1230 may calculate the target exercise speed proper to the user using at least on value showing the user's cardiorespiratory functional capacity.

The value showing the user's cardiorespiratory functional capacity may be maximal oxygen uptake (VO2max), and the user's maximal oxygen uptake (VO2max) may be calculated based on the checkup result value measured after the user performs the checkup of predetermined condition.

For example, user's maximal oxygen uptake (VO2max) may calculated based on time or distance measured by the user's running for a predetermined distance or time on an indoor exercise device according to the exercise checkup method as described in reference to FIG. 1 to FIG. 11.

And, the method by which the target speed calculation portion 1230 may convert the user's maximal oxygen uptake (VO2max) to the exercise speed is identical to that described in reference to FIG. 1 to FIG. 11, and a detailed description thereon will be omitted hereinafter.

On the other hand, information on the user's maximal oxygen uptake (VO2max) may be saved in the memory 1240 in advance, and information on the user's target exercise speed calculated at the target speed calculation portion 1230 may also be saved in the memory 1240.

The control portion 1250 may change color displayed on the display portion 1210 to show the corresponding user's exercise strength based on the user's current speed measured continuously through the speed measurement portion 1260 and the target exercise speed calculated through the target speed calculation portion 1270.

FIG. 17 is showing the exercise strength display method at the wearable device according to another embodiment of the present invention as a flow chart, and the depicted exercise strength display method will be described in connection to a block diagram showing an organization of the wearable device according to another embodiment of the present invention shown in FIG. 16.

Referring to FIG. 17, first, the target speed calculation portion 1270 of the wearable device 1200 may acquire information on the user's maximal oxygen uptake (VO2max) (S2110), and may calculate the user's target exercise speed using the acquired user's maximal oxygen uptake (VO2max) (S2120)

For example, the target speed calculation portion 1270 may read in information on the corresponding user's maximal oxygen uptake (VO2max) from the memory 1240 through the control portion 1250, and according to the exercise checkup method as described in reference to FIG. 1 to FIG. 11, may convert the user's maximal oxygen uptake (VO2max) to an exercise speed optimized thereto to provide.

The speed measurement portion 1260 may measure the user's current speed (v) which the user wears the wearable device 1200 and is exercising (S2130).

For example, the speed measurement portion 1220 may measure the user's speed continuously by a preset interval using a movement measurement sensor provided on the wearable device 1200.

After that, the control portion 1250 may change color displayed on the display portion 1210 to show the corresponding user's exercise strength based on the user's current speed measured continuously through the speed measurement portion 1260 and the user's target exercise speed calculated at the target speed calculation portion 1270 (S2140).

For example, the control portion 1250 may determine color displayed on the display portion 1210 according to the result in comparison of the target exercise speed calculated from the maximal oxygen uptake (VO2max) and the user's current speed.

In this case, the control portion 1250 may determine color displayed on the display portion 1210 according to whether the user's current speed (v) is in the target exercise strength range including the target exercise speed (V_target).

According to another embodiment of the present invention, the target exercise speed may be set as plurality each corresponding to plurality of exercise programs having different exercise strength one another.

For example, as described above in reference to FIG. 1 to FIG. 11, plurality of exercise programs may be proposed to the user including ‘beginner exercise mode’, ‘intermediate exercise mode’ and ‘advanced exercise mode’ according to the user's maximal oxygen uptake (VO2max).

In this case, the target exercise speed each corresponding to ‘beginner exercise mode’, ‘intermediate exercise mode’ and ‘advanced exercise mode’ may be set differently one another, and the target exercise speed may be increasing gradually in the order of ‘beginner exercise mode’, ‘intermediate exercise mode’, ‘advanced exercise mode’.

In case that the user selects ‘intermediate exercise mode’ from the plurality of exercise programs to perform running exercise, the target exercise speed may be calculated to 10.1 km/h according to the user's maximal oxygen uptake (VO2max), and accordingly range (v1˜v2) of the target exercise speed may be set to ‘9˜11 km/h’.

The wearable device 1200 according to an embodiment of the present invention may further comprise a vibration generation portion (not shown) or a voice output portion (not shown), and in case that the user's current speed is out of the target exercise strength range (or, out of the target exercise speed range), notice may be provided to the user by vibration or voice.

An embodiment of the present invention has been described above with an example that ‘proper’, ‘weak’ and ‘excessive’ of the user's current exercise strength is shown using ‘Blue’, ‘Green’ and ‘Red’, to which the present invention is not limited, but colors according to the current exercise strength may be changed or set by the user arbitrarily.

On the other hand, the control portion 1250 may verify the exercise program corresponding to the user's current speed (v) from plurality of exercise programs having different exercise strength one another, and may control the display portion 1210 to display color corresponding to the verified exercise program.

As described above, the wearable device 1200 have color corresponding to the user's current exercise strength be displayed through the display portion 1210, by which the user can verify easily visually the strength of the current exercise considering the maximal oxygen uptake (VO2max) of oneself.

On the other hand, in case that color displayed on the display portion 1210 is changed according to the user's current exercise strength, vibration or voice for notice of change of the exercise strength may be provided to the user through the vibration generation portion (not shown) or voice output portion (not shown) provided to the wearable device 1200.

According to another embodiment of the present invention, without setting the heart rate separately, the heart rate measured with progressing the running program at a speed suggested after measure checkup may be saved as a reference value, and a reference may be proposed by which proceed to next step based on the heart rate changed with progressing the corresponding exercise program.

For example, after checkup, the heart rate measurement with progressing the exercise program is recorded by user to be used to recognize the effect of train, by which without performing checkup again, the improvement of the user's exercise capacity may be understood.

And, while the running checkup is proceeded using the wearable device according to an embodiment of the present invention, the heart rate date measured together may be used as a value for determining whether the user performed the running checkup at the maximum capacity or low capacity value came out since not did the better though could.

For example, in the case that the heart rate measured through the checkup is under 80% of the heart rate reserve calculated by age, the corresponding checkup is determined as that the user did not do the maximum capacity, and an alarm corresponding thereto may be provided.

And a score about the user's recovery capacity may be proposed additionally based on the change (for example, slope) of the heart rate in which the heart rate increased after checkup drops toward stable time between 3 to 5 minutes after checkup end.

The method for providing exercise checkup service according to the present invention described above may be produced as a program for implemented on a computer and may be saved in a recording medium which the computer can read, and the examples of the recording medium which the computer can read may include ROM, RAM, CD-ROM, magnetic tape, floppy disk, light data storage device and the like, and further include ones realized in the form of carrier wave (for example a transmission through the internet).

The recording medium which the computer can read may be distributed over a computer system connected by a network, and the code which the computer can read may be saved and implemented in a distributed way. And, a functional program, a code and code segments for implementing the method may be inferred easily by programmers of technical field where the present invention pertains.

And, even though a desirable embodiment of the present invention has been illustrated and described above, the present invention may not be limited to the specific embodiment described above, but, of course, can allow a variety of changed implementation by a person having an ordinary understanding in the technical field where the present invention pertains without escaping from the gist of the present invention claimed in the claim, and these changed implementations should not be understood as to be separated from the technical idea or perspective of the present invention.

1. A method for providing exercise checkup service comprising
being inputted with a checkup result value measured after an examinee performing a checkup of a predetermined condition;
calculating at least one diagnosis reference value showing the examinee's cardiorespiratory functional capacity using the inputted checkup result value; and
classifying the examinee into one of plurality of groups based on at least one of the calculated diagnosis reference value to provide with information on an exercise program corresponding to the classified group.

2. The method for providing exercise checkup service according to claim 1, wherein
the checkup result value being time or distance measured after the examinee running on an indoor exercise device for a predetermined distance or time.

3. The method for providing exercise checkup service according to claim 2, wherein
being inputted with the checkup result value comprising receiving the checkup result value from the indoor exercise device using a wired or wireless communication method.

4. The method for providing exercise checkup service according to claim 1, further comprising
displaying at least one of the maximum oxygen uptake (VO2max) and oxygen utilization rate (Mets) calculated using the inputted checkup result value as a diagnosis reference value.

5. The method for providing exercise checkup service according to claim 1, further comprising
displaying a result of comparison of the calculated diagnosis reference value and the examinee's age average value as a ranking or percentile.

6. The method for providing exercise checkup service according to claim 1, wherein
the exercise program comprising plurality of exercise conditions according to different exercise strength or exercise step one another.

7. The method for providing exercise checkup service according to claim 1, wherein
the exercise program comprising an exercise condition according to illness information.

8. The method for providing exercise checkup service according to claim 1, wherein
each of the plurality of groups corresponding to each of plurality of exercise programs having different exercise conditions one another.

9. The method for providing exercise checkup service according to claim 6, wherein
the exercise condition comprising at least one of running speed and distance proposed to the examinee.

10. The method for providing exercise checkup service according to claim 1, further comprising
displaying information on each of a plurality of exercise programs corresponding to the plurality of groups.

11. The method for providing exercise checkup service according to claim 1, further comprising
displaying or outputting information on the exercise program corresponding to the inputted checkup result value, at least one of the calculated diagnosis reference value and the group in which the examinee being classified.

12. A recording medium recording a program for executing a method of claim 1 on a computer.

13. A terminal device for providing with an exercise checkup service, comprising
a user input portion being inputted with a checkup result value measured after an examinee performing a checkup of a predetermined condition; and
a display portion displaying at least one diagnosis reference value showing the examinee's cardiorespiratory functional capacity calculated using the inputted checkup result value and displaying information on an exercise program corresponding to a group of plurality of groups in which the examinee being classified based on the diagnosis reference value.

14. The terminal device according to claim 13, wherein
the plurality of groups being corresponding to each of plurality of exercise programs having different exercise conditions one another.

15. The terminal device according to claim 14, wherein
the exercise condition comprising running speed and distance proposed to the examinee.

16. The terminal device according to claim 13, wherein
the display portion displaying information on each of plurality of exercise programs corresponding to the plurality of groups.

17. A server device for providing with an exercise checkup service connected to a terminal device through a communication network, comprising
a communication portion receiving a checkup result value measured after an examinee performing a checkup of a predetermined condition from the terminal device;
a diagnosis portion calculating at least one diagnosis reference value showing the examinee's cardiorespiratory functional capacity using the received checkup result value, and classifying the examinee into one of plurality of groups based on at least one of the calculated diagnosis reference value;
a memory saving information on a plurality of exercise programs corresponding to each of the plurality of groups; and
a control portion organizing an exercise program corresponding to the group in which the examinee being classified using information saved in the memory and making information on an exercise condition included in the organized exercise program be transmitted to the terminal device through the communication portion.

18. The server device according to claim 17, wherein
the diagnosis portion calculating at least one of the examinee's maximum oxygen uptake (VO2max) and oxygen utilization rate (Mets) using the received checkup result value; and
at least one of the calculated examinee's maximum oxygen uptake (VO2max) and oxygen utilization rate (Mets) being transmitted to the terminal device through the communication portion to be displayed.

19. The server device according to claim 17, wherein
the organized exercise program comprising plurality of exercise conditions according to different exercise strength or exercise step one another.

20. The server device according to claim 17, wherein
the exercise condition comprising running speed and distance proposed to the examinee.
